(19) United States
(12) Patent Application Publication
(10) Pub. No.: US 2004/0040095 A1
(43) Pub. Date: Mar. 4, 2004

(54) Compositions and methods for decreasing color loss from dyed hair

(76) Inventors: Ginger King, Valley Cottage, NY (US); Xiaochun Luo, New City, NY (US); Robert E. Kalafsky, Ogdensburg, NJ (US); Philip Franco, Ocean Grove, NJ (US)

Correspondence Address: Charles N.J. Ruggiero, Ohlandt Greeley Ruggiero & Perle, One Landmark Square, 10th Floor, Stamford, CT, 06901-2682, US

(21) Appl. No.: 10/451,329

(22) PCT Filed: Dec. 22, 2000
(86) PCT No.: PCT/US00/35354

(51) Int. Cl. A61K 7/42

(52) U.S. Cl. 8/400; 424/59

(57) ABSTRACT

The present invention provides a novel hair composition, method and system for preventing color loss from dyed hair. The method of decreasing color loss from dyed hair includes a composition having a halogenated compound in an amount effective to decrease such color loss.

### BACKGROUND OF THE INVENTION

1. Field of the Invention

The present invention relates generally to hair care compositions. More particularly, the present invention relates to hair care compositions that preserve the color of artificially colored hair.

2. Description of the Prior Art

Hair may be artificially colored using permanent, demi-permanent and/or semi-permanent hair dyes. Hair dyes are used by a very broad spectrum of consumers. The manufacture of such hair dyes is a prominent industry.

Unfortunately, even “permanent” hair dyes do not last indefinitely, especially when the hair is washed with shampoo. In fact, even water alone can leach dye out of the hair. Obviously, this leaching effect is very undesirable to consumers. The loss of dye from the hair results in both color fade (less color intensity) and an overall color shift (due to the change in relative amounts of different dye molecules retained by each hair). Since the hair dying process is expensive, especially in salons, there is desire by consumers to effectively preserve the color of dyed hair.

Attempts to preserve the color of dyed hair have achieved varying success. For example, there are many hair care formulations containing one or more sunscreens. These types of formulations purport to prevent loss of hair color that is due to ultraviolet (UV) exposure. However, the primary cause for color loss from dyed hair is the daily washing, particularly shampooing, of hair. Thus, there is a need for compositions and methods that protect dyed hair from shampooing, as well as from UV exposure.

### SUMMARY OF THE INVENTION

It is an object of the present invention to provide a method that decreases color loss from dyed hair.

It is also an object of the present invention to provide a pre-wash treatment composition that decreases color loss from dyed hair.

It is another object of the present invention to provide a shampoo composition that decreases color loss from dyed hair.

These and other objects and advantages of the present invention are achieved by a composition comprising a halogenated compound in an amount effective to decrease color loss from dyed hair.

### DETAILED DESCRIPTION OF THE INVENTION

The present invention has, as one aspect, a hair composition having a halogenated compound. The halogenated compound functions as an active ingredient in a dyed hair color preservation complex to decrease color loss from dyed hair.

The halogenated compound is preferably a fluorinated compound. More preferably, the halogenated compound is a fluorogerbet. Preferably, the fluorogerbet is a cyclic fluorogerbet. A preferred cyclic fluorogerbet is dioctyidodecyl fluoroheptyl citrate. A preferred example of such a dioctyidodecyl fluoroheptyl citrate is available from Biosil under the tradename Biosil Basics Fluoro Guerbet 3.5. This preferred material is actually a blend of dioctyldodecyl fluoroheptyl citrate and cyclomethicone.

The halogenated compound is present in an amount effective to decrease color loss from dyed hair. Preferably, the halogenated compound is present in an amount effective to decrease such color loss of dyed hair due to washing, particularly with shampoos or other surfactant containing compositions. For example, the composition preferably includes about 0.0001 percent by weight (wt %) to about 5 wt %, more preferably about 0.0005 to about 2 wt %, of the halogenated compound, and most preferably from about 0.001 wt % to about 1 wt %. These ranges especially apply when the halogenated compound is dioctyldodecyl fluoroheptyl citrate.

It has been surprisingly discovered that when the halogenated compound is applied to dyed hair, color loss from the dyed hair due to wetting or washing is decreased. Even more surprisingly, it has been found that when the halogenated compound is applied to the hair as a pre-wash composition at a time before, but close to, washing the hair, such as shampooing, the loss of color from the dyed hair is dramatically decreased.

The composition of the present invention may also include a silicone polymer. Although it is preferred that the silicone polymer is present together with the halogenated compound to form a pre-wash composition, the silicone polymer may be applied as a separate washing composition, such as a shampoo, as will be described herein.

The silicone polymer is preferably an amodimethicone or a derivative thereof. Preferably, the amodimethicone is a trimethylsilylamodimethicone. Preferred trimethylsilyl-amodimethicones are available from GE Silicones under the tradenames SME253 and some 2115 as blends. SME253 contains C₁₁-C₁₅ Pareth-7, C₁₂-C₁₆ Pareth-9, and trimethylsilyl-amodimethicone. Another preferred example of such a blend is available from Dow Corning under the tradename DC 929.

When the silicone polymer is present, whether as part of the pre-wash composition or as a separate washing composition, the silicone polymer is preferably about 0.002 wt % to about 10 wt % or, more preferably, about 0.01 wt % to about 3 wt % based on the total weight of the composition. Most preferably, the amount of the silicone polymer, when present, is about 0.02 wt % to about 2 wt %.

The composition of the present invention may also include one or more antioxidants. The antioxidant may be included as part of the pre-wash composition or in the separate washing composition, or both.

The antioxidant is preferably a blend of two or more antioxidants. The present invention includes any antioxidant compatible with the composition. Examples of such antioxidants include, but are not limited to, sunflower seed extract, fennel extract, yeast and vitamin C (ascorbic acid), or any combination thereof. A preferred blend of antioxidants is available from Brooks Industries under the tradename Protection 3 Peroxisome. This blend includes the antioxidants fennel extract and yeast, as well as milk protein.

When present in either composition, the antioxidant is about 0.0001 to about 10 wt %, more preferably about 0.0003 wt % to about 5 wt %, and, most preferably, about 0.0002 wt % to about 4 wt %, based on the total weight of the composition containing such antioxidant.

Preferably, the compositions of the present invention also include one or more sunscreen agents. Preferably, the sunscreen agent protects against both UVA and UVB radiation. Examples of suitable sunscreen agents include, but are not limited to: oxybenzone, sulisobenzone, dioxybenzone, menthyl anthranilate, para aminobenzoic acid, dea methoxycinnamate, octyl methoxycinnamate, octocrylene, drometrizole trisiloxane, octyl salicylate, homomenthyl salicylate, octyl dimethyl PABA, TEA salicylate, titanium dioxide, zinc oxide, butylmethoxydibenzoylmethane, methyl benzilidene camphor, octyl triazone, terepthalydiene, dicamphor sulfonic acid, ethyl PABA, hydroxy methylphenyl benzotriazole, methylene bisbenzotriazoyltetramethylbutylphenol (MBBT), bis-ethylhexyl oxyphenol methoxyphenol triazine (BEMT), cinnamidopropyltrimethyl ammonium chloride, dimethyl PABA amidolaurdimmonium tosylate, and any suitable mixture thereof. The preferred sunscreens are oxybenzone, sulisobenzone, octyl methoxycinnamate, cinnamidopropyltrimethyl ammonium chloride, and dimethyl PABA amidolaurdimmonium tosylate. The sunscreen may be added to the compositions of the present invention with any combination of the aforementioned ingredients.

The compositions of the present invention preferably include a film former. The film former may be any film forming compound compatible with the composition and with the intended purpose of the composition. One preferred example of a film former is milk protein. In particular, when the compositions of the present invention include an antioxidant, it is preferred that they also include a film former.

The present invention also includes a method of decreasing color loss of dyed hair. The method includes the step of applying to dyed hair a pre-wash composition having the halogenated compound in an amount effective and as an active ingredient to decrease color loss. Preferably, this composition contains indicia on the package, container or other instructions, to apply before the dyed hair is washed with a separate washing composition. The pre-wash composition may be allowed to “set” for as little as 10 seconds, preferably about one (1) minute or more, more preferably about three (3) minutes. The halogenated compound may also be present for further application to the hair in the separate washing composition. This separate composition also includes the silicone polymer. Preferably, this separate composition also includes an antioxidant. It may further include the film-former and/or sunscreen.

### EXAMPLE 1

The effect of the present invention against UV exposure was assessed by exposing four hair swatch samples to light in a “fadeometer” that simulates the effects of four days of exposure to direct sunlight. First, a swatch of hair was dyed red. Hair dyed red is particularly susceptible to color loss. From this swatch of dyed hair, four swatches were separated and then treated as follows.

A first swatch served as a positive control and was not subjected to the fadeometer. A second swatch served as a negative control and was both wetted with deionized water and subjected to the fadeometer. The second swatch (negative control) showed a 50% color intensity difference as visually compared to the first swatch (positive control). A third swatch treated with an aqueous solution having 5 wt % halogenated compound, namely dioctyldodecyl fluoroheptyl citrate, showed a 25% color intensity difference as visually compared to the positive control. Surprisingly, a fourth swatch treated with a composition including dioctyldodecyl fluoroheptyl citrate, trimethylsilyl-amodimethicone, and an antioxidant blend of fennel extract, yeast and milk protein resulted in a 10% color intensity decrease as visually compared to the positive control swatch and, in fact, had a more intense red shade.

### EXAMPLE 2

The effect of the present invention in a hair wash treatment was also investigated. Again, a swatch of hair was dyed red and separated to provide the swatches used for the tests below.

A first dyed swatch was untreated and served as the positive control. A second dyed swatch served as the negative control and was subjected to five (5) washings using a conventional shampoo. As visually compared to the first (positive control) dyed swatch, the second (negative control) dyed swatch demonstrated a 50% shade difference.

A third dyed swatch washed with a shampoo containing (1) the silicone polymer, namely trimethylsilyl-amodimethicone, and (2) the halogenated compound, namely dioctyldodecyl fluoroheptyl citrate, resulted in a 20% shade difference after 5 washings.

Finally, a fourth dyed swatch washed with a shampoo containing (1) the same preferred blend of antioxidants, namely fennel extract, yeast, and milk protein, and (2) dioctyldodecyl fluoroheptyl citrate, resulted in only a 10% shade difference after five washings as visually compared to the positive control.

### EXAMPLE 3

The effect of the present invention in a hair pre-wash treatment was also investigated. Again, a swatch of hair was dyed red and separated to provide the swatches used for the tests below.

A first dyed swatch was untreated and served as the positive control. A second dyed swatch served as the negative control and was subjected to seven (7) washings using a conventional shampoo. As visually compared to the first (positive control) dyed swatch, the second (negative control) dyed swatch demonstrated a 50% shade difference.

A third dyed swatch pretreated with the halogenated compound, namely dioctyldodecyl fluoroheptyl citrate, resulted in a 15% shade difference after seven (7) washings with a conventional shampoo, as visually compared to the positive control swatch.

Thus, treatment of dyed hair with the present invention provides significant improvement in color retention.

Although the present invention has been discussed with particular attention to dyed hair, it is believed that the present invention may be used to decrease the loss of dyes and, thus, color from other keratin-containing areas, such as skin and nails.

The present invention having been thus described with particular reference to a preferred form thereof, it will be obvious that various changes and modifications may be made therein without departing from the spirit and scope of the present invention as defined in the appended claims.

We claim:

1. A method of decreasing color loss from dyed hair comprising the step of applying to said dyed hair a composition having a halogenated compound in an amount effective and as an active ingredient to decrease the color loss.

2. The method of claim 1, wherein the halogenated compound is a fluorinated compound.

3. The method of claim 1, wherein the halogenated compound is a fluorogerbet.

4. The method of claim 1, wherein the halogenated compound is a cyclic fluorogerbet ester.

5. The method of claim 1, wherein the halogenated compound is dioctyl fluoroheptyl citrate.

6. The method of claim 1, further comprising the step of subsequently applying a shampoo to said hair.

7. The method of claim 6, wherein said shampoo comprises a silicone polymer and an antioxidant.

8. The method of claim 7, wherein said silicone polymer is an amodimethicone or derivative thereof.

9. The method of claim 7, wherein said shampoo further comprises the halogenated compound.

10. The method of claim 7, wherein said shampoo further comprises a film former.

11. The method of claim 6, wherein said composition is applied one or more minutes before said shampoo.

12. The method of claim 1, wherein said composition further comprises a sunscreen agent.

13. The method of claim 1, wherein said composition further comprises an antioxidant.

14. The method of claim 1, wherein said composition further comprises a silicone polymer.

15. The method of claim 13, wherein said composition further comprises a silicone polymer.

16. The method of claim 13, wherein said composition further comprises a film former.

17. A pre-wash composition comprising a halogenated compound in an amount effective and as an active ingredient to decrease color loss from dyed hair, and associated indicia for application of the composition onto hair before shampooing.

18. The pre-wash composition of claim 17, wherein the halogenated compound is a fluoroguerbet ester.

19. The pre-wash composition of claim 17, wherein the halogenated compound is a cyclic fluoroguerbet ester.

20. The pre-wash composition of claim 17, wherein the halogenated compound is dioctyl fluoroheptyl citrate.

21. The pre-wash composition of claim 17, further comprising a sunscreen agent.

22. The pre-wash composition of claim 17, further comprising an antioxidant.

23. The pre-wash composition of claim 17, further comprising a silicone polymer.

24. The pre-wash composition of claim 22, further comprising a silicone polymer.

25. A hair care composition comprising a dyed hair color preservation complex having a halogenated compound, a silicone polymer, and an antioxidant, wherein said complex is present in an amount effective to decrease color loss from dyed hair.

26. The hair care composition of claim 25, further comprising a film former.

27. The hair care composition of claim 25, further comprising a sunscreen agent.

28. A hair care system for decreasing color loss from dyed hair comprising:
a first composition having a halogenated compound in an amount effective and as an active ingredient to decrease the color loss of dyed hair; and
a second composition having a silicone polymer and an antioxidant.
